(19) United States
(12) Patent Application Publication
(10) Pub. No.: US 2006/0073107 A1
(43) Pub. Date: Apr. 6, 2006

(54) Use of vitamin D3 (cholecalciferol) in sunscreens

(76) Inventor: John Robert Person, Charlton, MA (US)

Correspondence Address: John Robert Person, 50 Casey Rd., PO Box 396, Charlton City, MA, 01508, US

(21) Appl. No.: 10/956,993

(22) Filed: Oct. 4, 2004

(51) Int. Cl. A61K 31/59; A61K 8/63

(52) U.S. Cl. 424/59; 514/167

(57) ABSTRACT

Despite the widespread availability of highly-protective sunscreens, there is a worldwide epidemic of skin cancer. A possible reason for this is that sunscreens, while limiting damage to the DNA, may promote cancer growth by preventing vitamin D synthesis in the skin. To have biologic effects, Vitamin D must be converted to its active form, calcitriol, in a two-step process formerly thought to occur first in the liver and secondly in the kidney. It is now known, however, that this conversion can be accomplished entirely in the skin. Cutaneous activation of Vitamin D by ultraviolet light puts the potent anti-tumor effects of calcitriol at the site of tumor formation and in significant concentration, a fact seemingly ignored by workers in the field. A similar effect may be achieved by incorporating vitamin D into sunscreens. Because calcitriol also promotes cellular growth and differentiation, this also may be of benefit for photoaging.

### BACKGROUND

It is common knowledge that we are experiencing an epidemic of skin cancer. Non-melanoma (basal and squamous cell carcinoma) skin cancer has increased by 3-8% per year since the 1960's (Glass A G et al), and the lifetime risk of malignant melanoma has increase from 1/500 in 1960 (Kopf A W et al) to 1/75 (estimated) in 2000 (Rigel D S). High SPF (sun-protective-factor, essentially a measure of protection against sunburn or ultraviolet B) sunscreens have been available for almost 30 years and broader spectrum (which also block some longwave ultraviolet) for almost 15 years. There is some evidence that vigorous use of sunscreens may reduce the incidence of nonmelanoma cancer (Green A et al; Naylor M F et al), but is difficult to reconcile this with the skin cancer epidemic. The situation with melanoma is less clear; with some studies showing a protective effect and other showing that sunscreens may increase the risk of melanoma (see review, Bastuji-Garin S et al).

There are no adequate animal models for basal cell carcinoma or melanoma.

Among the various hypotheses put forth to explain the failure of sunscreens, especially in the prevention of melanoma, is that sunscreens inhibit epidermal synthesis of vitamin D₃ (cholecalciferol), and that this promotes the growth of cancer (see review, Osborne J E et al). Vitamin D₃ is synthesized by epidermal keratinocytes on exposure to UVB, but must undergo activation first by 25-hydroxylation and then 1-alpha hydroxylation to convert it to 1,25 dihydroxyvitamin D₃, or calcitriol, the active form of vitamin D. Traditionally, these conversions have been thought to occur in the liver and kidney exclusively (Osborne J E et al). Calcitrol is a potent regulator of cell growth (Kawa S et al) and differentiation (Hosomi J et al) has an inhibitory effect on cellular death (Park W H et al; McGuire T F et al) and new blood vessel growth (i.e., into tumors) (Mantell D J et al; Majewski S et al). Low vitamin D levels have been associated with breast, prostate, and colon cancer (Osborne et al). New vitamin D analogues have been shown to be very effective in preventing chemical tumorgenesis in mice (Kensler et al).

Despite the traditional dogma that hepatic and renal activation are necessary to produce calcitriol, it has been known for many years that the skin is capable of converting vitamin D₃ to calcitriol on its own (Bikle D D, Nemanic M K, Whitney J D, et al; Bikle D D, Nemanic M K, Gee E et al, Matsumoto K et al, Schuessler et al) the scientific community has not recognized the implications of these findings, perhaps because of the negative report by MacLaughlin et al. As an example, note the recent studies showing normal calcitriol serum levels (Cornwell M L et al), normal 25-hydroxyvitamin D₃ serum levels (Reichrath J et al), and normal dietary vitamin D intake (Weinstock M A et al) in melanoma patients. Serum vitamin D levels and dietary intake are of little importance if the epidermis can generate calcitriol. Hence this application is contrarian. Activation of vitamin D₃ to calcitriol within the epidermis puts the anti-tumor activity of calcitriol at the site of tumor formation in high concentration. Most of the calcitriol remains within the keratinocyte (Matsumoto K et al), a fact that is probably obvious, since individuals who sunbathe do not experience elevated calcium levels (an obvious effect of both topical and circulating calcitriol). This might then explain why protection against sunburn seems not to be protection against skin cancer.

Melanoma cells express the vitamin D receptor and calcitriol inhibits their growth (Colsten K et al, Evans S R et al) and invasion (Yudon et al). Melanoma cells can activate vitamin D₃ (Frankel T L et al), but it is not known if normal melanocytes can accomplish this, or whether melanocytes can synthesize vitamin D₃. Polymorphisms of the vitamin D receptor are associated with an increased susceptibility to and worsened progress in melanoma (Hutchinson R E et al; Halsall J A et al).

Because of its effects on growth and differentiation, vitamin D and its analogs are of potential benefit in the treatment of photoaging. The published data are meager (see Nagpal S et al), but there have been several patents granted for vitamin D₃ analogs (U.S. Pat. Nos. 5,747,479, 5,804,574, 5,811,414) or vitamin D₂ (U.S. Pat. Nos. 5,476,661, 5,776,461), but none for vitamin D₃ itself. (The sole patent I could find for vitamin D₃ itself was for its new use in itching, U.S. Pat. No. 5,789,399).

Vitamins A, E, and C are currently added to some sunscreens.

Topical vitamin D has been available over-the-counter for many decades as Schering-Plough's “A&D Ointment,” a preparation for diaper rash and thus whose safety speaks for itself. It contains approximately the adult recommended daily allowance of vitamin D (10 mcg or 400 units) per ounce (about the amount necessary to cover an adult body). (The exact formulation cannot be discerned from the labeling, but cod liver oil, about 400 units of vitamin D per teaspoon, is the first-listed of the 31.1% “inert” ingredients).

### REFERENCES

Bastuji-Garin S, et al. Cutaneous malignant melanoma, sun exposure, and sunscreen use: epidemiological evidence Brit J Dermatol 2002; 146 (suppl. 61) 24-30 (review of the controversy).
Bikle D D, Nemanic M K, Gee E, et al. 1,25 dihydroxyvitamin D₃ production by human keratinocytes. Kinetics and regulation. J Clin Invest 1986; 557-66.
Bikle D D, Nemanic M K, Whitney J D et al. Neonatal human foreskin Keratinocytes produce 1,25 dihydroxyvitamin D₃. Biochemistry 1986; 25:1545-8.
Colston K, et al. 1,25-Dihydroxyvitamin D₃ and malignant melanoma: the presence of receptors and inhibition of cell growth in culture. Endocrinology 1981; 108:1083-6.
Cornwell M L, et al. Prediagnositc serum levels of 1,25 dihydroxyvitamin D and malignant melanoma. Photodermatol Photoimmunol Photomed 1992; 9:109-12.
Evans S R, et al. Vitamin D receptor and growth inhibition by 1,25 dihydroxyvitamin D₃ in human malignant melanoma cell lines. J Surg Res 1996; 61:127-33.
Glass A G, et al. The emerging epidemic of melanoma and squamous cell skin cancer. JAMA 1989; 262:2097-100.
Green A, et al. Daily sunscreen application and beta carotene supplementation in the prevention of basal-cell and squamous-cell carcinoma of the skin: a randomized controlled trial. Lancet 1999; 359:723-9.
Halsall J A, et al. A novel polymorphism in the IA promoter region of the vitamin D receptor is associated with altered susceptibility and prognosis in malignant melanoma. Br J Cancer 2004; 16:765-70.
Hosomi J, et al. Regulation of terminal differentiation of cultured epidermal cells by 1 alpha, 0.25 dihydroxyvitamin D₃. Endocrinology 1983; 113:1950-7.
Hutchinson P E, et al. Vitamin D receptor polymorphisms are associated with altered prognosis in patients with melanoma. Clin Cancer Res 2000; 6:498-504.
Kawa S, et al. Vitamin D analogues up regulate p21 and p27 during growth inhibition of pancreatic cancer cell lines. Br J Cancer 1997; 76:884-9.
Kersler T W, et al. Conceptually new deltanoids (vitamin D analogs) inhibit skin tumor genesis. Carcinogenesis 2000; 21:1341-5.
Kopf A W, et al. The rising incidence and mortality rate of malignant melanoma. J Dermatol Surg Oncol 1982; 8:760-1.
MacLaughlin et al. Cultured Keratinocytes cannot metabolize vitamin D sub 3 to 25 hydroxyvitamin D sub 3. Feder Eruo Biochem Soc 1991; 28:909-11.
Majewski S, et al. Vitamin D₃ is a potent inhibitor of tumor cell-induced angiogenesis. J Invest Dermatol Symp Proc 1996; 1:97-101.
Mantell D J, et al. 1 alpha, 25-Dihydroxyvitamin D₃ inhibits angiogenesis in vitro and in vivo. Circ Res 2000; 87:214-20.
Matsumoto K, Azuma Y, Kiyoki M, et al. Involvement of endogenously produced 1,25 dihydroxyvitamin D3 in the growth and differentiation of human Keratinocytes. Biochim Biophys Acta 1997; 1092:311-8.
McGuire T F, et al. Vitamin D₃—induced apoptosis of murine squamous cell carcinoma cells. Selective induction of capsase-dependent MEK cleavage and upregulation of MEKK-1. J Biol Chem 2001; 276:263-73.
Miller D L, et al. Nonmelanoma skin cancer in the United States: Incidence. J Am Acad Dermatol 1994; 30:774-8.
Nagpal S, et al. Vitamin D analogs: mechanism of action and therapeutic applications. Curr Med Chem 2001; 8:1661-79.
Naylor M, et al. High sun protective factor (SPF) sunscreens in the suppression of actinic neoplasia. Arch Dermatol 1995; 131:170-5.
Osborne J E, et al. Vitamin D and systemic cancer: is this relevant to malignant melanoma? Brit J Dermatol 2002; 147:197-213 (excellent review and hypothesis).
Park W H, et al. Induction of apoptosis by vitamin D3 analogue EB1089 in NCI-H929 myeloma cells via activation of capsase-3 and p38 MAP Kinase. Br J Hematol 2000; 109:576-83.
Reichrath J, et al. No evidence for reduced 25-hydroxyvitamin b serum levels in melanoma patients. Cancer Causes Control 2004; 15:97.
Rigel D S. The gender-related issues in malignant melanoma. Hawaii Med J 1993; 52:124-46.
Schuessler M, Astecker N, Herzig G, et al. Skin is an automatous organ in synthesis, two-step activation and degradation of vitamin D (3): CYP27 in epidermis completes the set of essential vitamins D (3) hydroxylases. Steroids 2001; 66:399-408.
Weinstock M A, et al. Case-control study of melanoma and dietary vitamin D: implications for advocacy of sun protection and sunscreen use. J Invest Dermatol 1992; 98:809-11.

### FIELD OF INVENTION

The addition of up to 5% vitamin D₃, cholecalciferiol, or equivalent concentration of cod liver oil, in any lipid-containing sunscreen for the prevention of skin cancer, eradication of precancers, or treatment of photoaging.

What is claimed is:

1. A new use method of potentially preventing skin cancer and eradicating precancers by the addition of up to 5% cholecalciferol (vitamin D₃) to topical sunscreens.

2. A new use method of potentially reducing photoaging by the addition of up to 5% cholecalciferol (vitamin D₃) to topical sunscreens.
